(19) United States
(12) Patent Application Publication
(10) Pub. No.: US 2026/0248579 A1
(43) Pub. Date: Aug. 27, 2026

(54) STOWAGE COVERS FOR ROBOTIC SURGICAL SYSTEMS

(71) Applicant: Cilag GmbH International, Zug (CH)

(72) Inventor: Richard LUM, Blue Ash, OH (US)

(73) Assignee: Cilag GmbH International, Zug (CH)

(21) Appl. No.: 19/064,494

(22) Filed: Feb. 26, 2025

(51) Int. Cl. A61B 46/10; A61B 46/00

(52) U.S. Cl. CPC A61B 46/10; A61B 46/40

(57) ABSTRACT

A stowage cover is disclosed that includes an elongate body including opposing proximal and distal ends, a first opening defined at the proximal end and sized to receive a robotic arm of a robotic surgical system into an interior of the body, a second opening defined in the body between the proximal and distal ends, and a fastener coupled to the body and operable to secure the body to the robotic arm once the body is received within the interior of the body.

### BACKGROUND

The present disclosure relates to surgical systems and, more particularly, to stowage covers for robotic arms of robotic surgical systems.

Robotic-assisted surgery has gained prominence over the past few decades, offering enhanced precision, flexibility, and control for surgeons. Robotic surgical systems have been used in various medical specialties including urology, gynecology, cardiology, and general surgery. These systems feature robotic arms that perform surgical procedures with high accuracy, often through minimally invasive methods. However, as these robotic arms are used in diverse medical environments, they face certain challenges that need to be addressed to improve safety, hygiene, and overall functionality.

As one example, after a surgical procedure, the robotic arms are commonly retracted and stowed under the operating table. When a patient is transferred from the operating table to a gurney, there is a chance that various fluids may leak onto the stowed robotic arms, which may compromise the sterility of the operating room and affect the long-term functionality of the robotic arms. Exposure to fluids can also potentially lead to malfunctions or wear and tear.

To mitigate these issues, there is a growing demand for covers that can provide a protective barrier for robotic surgical arms when not in use. Despite the widespread use of robotic surgical systems, there is currently a lack of specialized protective covers that can effectively meet needed requirements. Existing solutions, such as generic covers or drapes, are either inadequate in terms of durability and functionality or do not address the specific needs of the robotic system.

Accordingly, improved stowage covers for robotic surgical systems are desired.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 illustrates an example robotic surgical system that may incorporate the principles of the present disclosure.

FIG. 2 illustrates an alternative view of the robotic surgical system of FIG. 1.

FIG. 3 is a schematic diagram of an improved stowage cover for the robotic surgical system of FIG. 1 in an unfolded state, in accordance with at least one aspect of the present disclosure.

FIG. 4 is a detailed view of a mesh vent assembly of the stowage cover of FIG. 3, in accordance with at least one aspect of the present disclosure.

FIG. 5 is a detailed view of a fastener of the stowage cover of FIG. 3, in accordance with at least one aspect of the present disclosure.

FIG. 6 is a detailed view of an identification component of the stowage cover of FIG. 3, in accordance with at least one aspect of the present disclosure.

FIG. 7 are symbols provided on the stowage cover of FIG. 3, in accordance with at least one aspect of the present disclosure.

FIG. 8 is the stowage cover of FIG. 3 in a partially-folded state, in accordance with at least one aspect of the present disclosure.

FIG. 9 is the stowage cover of FIG. 8 with an opening thereof exposed, in accordance with at least one aspect of the present disclosure.

FIG. 10 is the stowage cover of FIG. 3 in a folded state, in accordance with at least one aspect of the present disclosure.

FIG. 11 depicts the stowage cover of FIG. 3 in the process of being positioned over a robotic arm of the robotic surgical system of FIG. 1, in accordance with at least one aspect of the present disclosure.

FIG. 12 is the stowage cover of FIG. 3 fully positioned over a robotic arm of the robotic surgical system of FIG. 1, in accordance with at least one aspect of the present disclosure.

FIG. 13 is an enlarged view of a portion of the fully positioned stowage cover of FIG. 12, in accordance with at least one aspect of the present disclosure.

### DETAILED DESCRIPTION

The present disclosure relates to surgical systems and, more particularly, to stowage covers for robotic arms of robotic surgical systems.

Disclosed herein are stowage covers comprising non-sterile, single-use devices configured to protect the robotic arms of robotic surgical systems during patient transfer and when the surgical system is not in use. As described herein, the stowage covers may be advantageous in preventing various fluids from contacting the robotic arms when the arms are stowed, thus ensuring that the system meets the IPX4 table rating (a rating indicative of protection against splashes of fluids) in the stowed pose (position). Each stowage cover is configured to fully cover one robotic arm, and four stowage covers may be used to fully cover an example robotic surgical system with four arms. As described herein, the stowage covers can be packaged in a telescopic folded configuration that enables the user to efficiently deploy and install the stowage cover. The stowage covers are installed when the arm is deployed, and they allow the arms to move from procedure poses to stow pose unhindered.

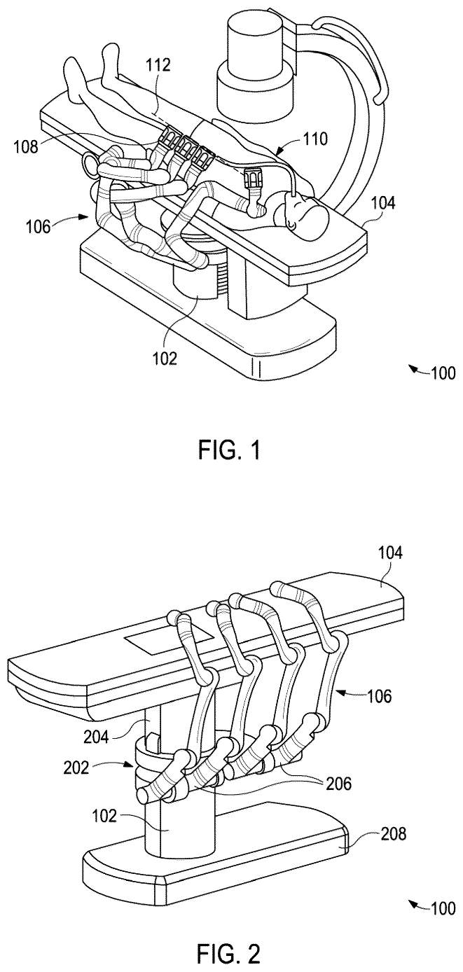

FIG. 1 illustrates an example robotic surgical system 100 that may incorporate the principles of the present disclosure. As illustrated, the system 100 may be configured for a bronchoscopy procedure and includes a support structure or column 102 for supporting a platform 104 (shown as a “table” or “bed”) over the floor. The system 100 includes one or more robotic arms 106 (four shown), and each robotic arm 106 includes an instrument driver 108 designed to manipulate an elongated medical instrument, such as a bronchoscope 110, through or along a virtual rail 112 formed from the linear alignment of the instrument drivers 108. In practice, a C-arm for providing fluoroscopic imaging may be positioned over the patient's upper abdominal area by placing the emitter and detector around the table 104. As will be appreciated, the principles of the present disclosure are not limited to the particular configuration of the system 100, but are equally applicable to other types and configurations of robotic surgical systems configured for various types of surgical procedures.

FIG. 2 provides an alternative view of the system 100 without the patient and medical instrument for discussion purposes. As shown, the column 102 may include one or more carriages 202 shown as ring-shaped in the system 100, from which the one or more of the robotic arms 106 may be based. The carriages 202 translate along a vertical column interface 204 that runs the length (height) of the column 102 to provide different (vertical) vantage points from which the robotic arms 106 may be positioned to reach the patient. The carriage(s) 202 may rotate around the column 102 using a mechanical motor positioned within the column 102 to allow the robotic arms 106 to have access to multiples sides of the table 104, such as, for example, both sides of the patient. In embodiments with multiple carriages 202, the carriages 202 may be individually positioned on the column 102 and may translate and/or rotate independent of the other carriages 202. While carriages 202 need not surround the column 102 or even be circular, the ring-shape as shown facilitates rotation of the carriages 202 around the column 102 while maintaining structural balance. Rotation and translation of the carriages 202 allows the system 100 to align medical instruments, such as endoscopes and laparoscopes, into different access points on the patient.

In other embodiments, the system 100 can include a patient table or bed with adjustable arm supports in the form of bars or rails extending alongside it. The robotic arms 106 can be attached to the adjustable arm supports (e.g., via a shoulder with an elbow joint), which can be vertically adjusted. By providing vertical adjustment, the robotic arms 106 are advantageously capable of being transitioned from an operating state, as shown in FIGS. 1 and 2, to a stowed state (not shown) in which the robotic arms 106 are stowed compactly beneath the patient table or bed.

The arms 106 may be mounted on the carriages 202 through a set of arm mounts 206 comprising a series of joints that individually rotate and/or telescopically extend to provide additional configurability to the robotic arms 106. Additionally, the arm mounts 206 are positioned on the carriages 202 such that when the carriages 202 are appropriately rotated, the arm mounts 206 may be positioned on either the same side of the table 104 (as shown in FIG. 2), on opposite sides of table 104 (not shown), or on adjacent sides of the table 104 (not shown).

The column 102 structurally provides support for the table 104, and a path for vertical translation of the carriages 202. Internally, the column 102 may be equipped with lead screws for guiding vertical translation of the carriages, and motors to mechanize the translation of said carriages based the lead screws. The column 102 may also convey power and control signals to the carriage 202 and robotic arms 106 mounted thereon.

A table base 208 houses heavier components to balance the table/bed 104, the column 102, the carriages 202, and the robotic arms 106. The table base 208 may also incorporate rigid casters to provide stability during procedures. Deployed from the bottom of the table base 208, the casters extend in opposite directions on both sides of the base 208 and retract when the system 100 needs to be moved.

Additional information regarding the robotic surgical system 100 can be found in U.S. patent application Ser. No. 18/513,409, entitled “MEDICAL INSTRUMENT ACTUATOR OPERATION”, which published on Mar. 14, 2024 as U.S. Pat. Pub. No. 2024/0081930, the contents of which are hereby incorporated by reference in their entirety herein.

As referenced elsewhere herein, robotic arms, like robotic arms 106, are used in diverse medical environments and face certain challenges that need to be addressed to improve safety, hygiene, and overall functionality. One such solution is applying a stowage cover to the robotic arms, which can provide a protective barrier for the arms when not in use. However, despite the widespread use of robotic surgical systems, there is currently a lack of specialized protective covers that can effectively meet needed requirements. Existing solutions, such as generic covers or drapes, are either inadequate in terms of durability and functionality or do not address the specific needs of the robotic system. Accordingly, improved stowage covers for robotic surgical systems are desired.

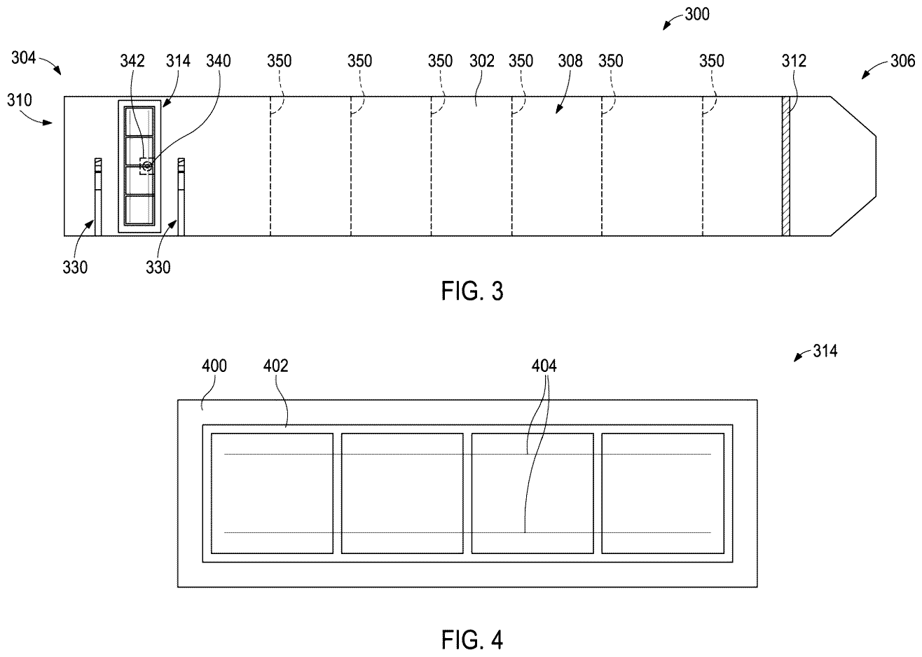

FIG. 3 is a schematic plan view of an example stowage cover 300, in accordance with at least one aspect of the present disclosure. The stowage cover 300 is shown in its fully deployed and unfolded configuration to enable viewing of various component parts thereof. As illustrated, the stowage cover 300 comprises an elongate body 302 that includes a first or “proximal” end 304, a second or “distal” end 306, a first lateral side 308, and a second lateral side 1000 (FIG. 10) opposite the first lateral side 306. The body 302 may comprise a tubular sleeve structure such that the first and second lateral sides 308, 1000 comprise arcuate portions of the outer perimeter of the body 302 that are angularly offset from each other by approximately 180°. The body 302 is sealed (closed) at the distal end 306 and defines an opening 310 (shown more clearly in FIG. 9) at the proximal end 304 to receive one of the robotic arms 106 (FIG. 1) therethrough and into an interior of the body 302.

The body 302 may be made of a variety of materials including, but not limited to, a polymer, such as Polyester Thermoplastic Polyurethane (TPU), Linear Low Density Polyethylene (LLDPE), Low-Density Polyethylene (LDPE), High-Density Polyethylene (HDPE), Polypropylene (PP), Ethylene-Vinyl Acetate (EVA), Polyethylene Terephthalate (PET), Polybutylene (PB), or Polycarbonate (PC), or combinations thereof.

In some embodiments, the stowage cover 300 may include a visual indicator 312 that enables a user to visually identify the approximate location of the distal end 306 of the body 302. Accordingly, in at least one embodiment, the visual indicator 312 may be arranged at or near the distal end 306. The visual indicator 312 may include a strip of tape (such as blue tape) adhered to the body 302, an adhesive, or a symbol printed onto the body 302.

In some embodiments, as illustrated, the stowage cover 300 may include a mesh vent assembly 314 located on the first lateral side 308 of the body 302 at a location between the proximal and distal ends 304, 306, such as adjacent the proximal end 304. With reference to FIG. 4, the mesh vent assembly 314 may include a base layer 400 and a mesh vent 402 positioned on and coupled to the base layer 400, such as with an adhesive, via laser welding, with heat-welding, or heat sealing. One or more openings 404 may be defined in the base layer 400. In at least one embodiment, as illustrated, the openings 404 may comprise slits through the base layer 400. Alternatively, the openings 404 may comprise a plurality of apertures, such as circular through holes, through the base layer 400. The openings 404 may extend about a portion of the circumference of the body 302 and otherwise substantially perpendicular to the sidewall of the body 302.

The base layer 400 may be made of the same materials as the body 302 (FIG. 3), such as Polyester Thermoplastic Polyurethane (TPU) or Linear Low Density Polyethylene (LLDPE), or may be comprised of a different material than the body 302 (FIG. 3), such as Low-Density Polyethylene (LDPE), or any other material listed elsewhere herein.

In contrast, the mesh vent 402 may be made of a polymer, such as Polyethylene (PE), Nylon, Polyester, Polyethylene Terephthalate (PET), Polycarbonate (PC), Polyoxymethylene (POM), Polypropylene (PP), Aramids (e.g., Kevlar, Nomex), Polyetheretherketone (PEEK), or Polyurethane (PU), or any combination thereof. The mesh vent 402 may be coupled to the base layer 400 such that the mesh vent 402 is situated within the interior of the body 302 (FIG. 3). Alternatively, the mesh vent 402 may be coupled to the base layer 400 such that the mesh vent 402 is situated on an exterior side of the body 302 (FIG. 3). Alternatively, the mesh vent assembly 314 may include two (first and second) mesh vents 402 coupled to opposing sides of the base layer 400 such that the first mesh vent 402 is situated on an exterior side of the body 302 (FIG. 3) and the second mesh vent 402 is situated within the interior of the body 302 (FIG. 3). The mesh vent 402 may include a plurality of apertures that allow air and fluid flow therethrough, but that provides a barrier to dust, dirt, and other debris of a predetermined size.

Referring now to both FIGS. 3 and 4, in some embodiments, the mesh vent assembly 314 may be constructed separate from the body 302 and then coupled (attached) thereto. For instance, the body 302 may define an aperture (not shown) on the first lateral side 308 sized to receive the mesh vent assembly 314 therein. With the mesh vent assembly 314 situated in the aperture, the base layer 400 may be coupled to the body 302. For instance, the base layer 400 may be coupled to the body 302 around its perimeter thereof using any suitable fastening technique, such as thermal fastening, ultrasonic welding, or with an adhesive (e.g. tape or glue), to seal the edges of the base layer 400 to the body 302.

In alternative embodiments, the base layer 400 and the body 302 may be a uniform component (i.e. the base layer 400 is a portion of the body 302). In such embodiments, the openings 404 may be defined in the base layer 400 of the body 302, such as with a knife or any other suitable cutting tool, and then the mesh vent 404 may be coupled to the body 302 over the openings 404. In other alternative embodiments, the base layer 400 may be welded (affixed) to a plastic frame that is then welded (affixed) to the body 302.

The stowage cover 300 may further include a plurality of fasteners 330 operable to help secure the stowage cover 300 to the robotic arm 106 (FIG. 1). For instance, the stowage cover 300 may include a first fastener 330 located between the proximal end 304 of the body 302 and the mesh vent assembly 314 and a second fastener 330 located between the distal end 306 of the body 302 and the mesh vent assembly 314.

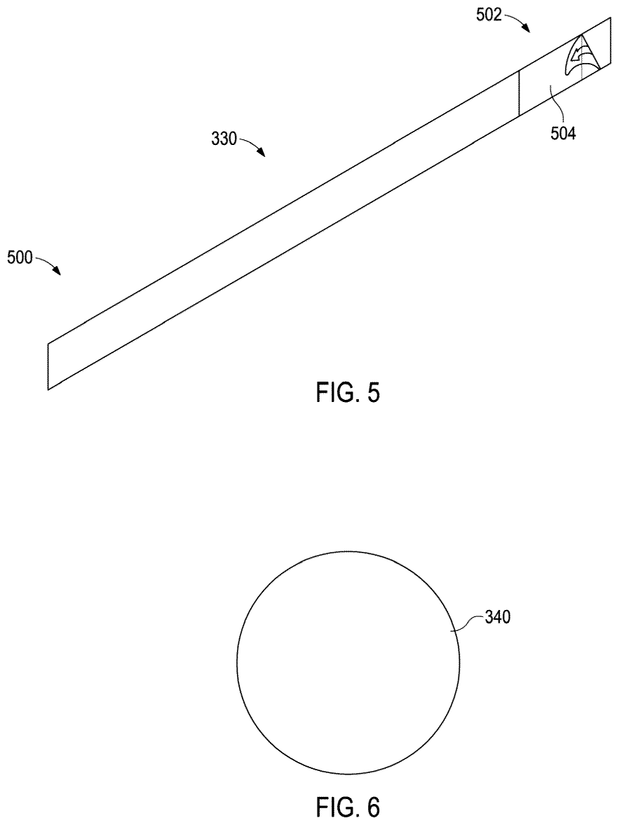

Referring to FIGS. 3 and 5, illustrated is an enlarged isometric view of one of the first and second fasteners 330, according to one or more embodiments. As illustrated, the fastener 330 may comprise a repositionable wrap tape that includes a first end 500 and a second end 502. The first end 500 may include a first adhesive that permanently adheres the first end 550 to the body 302; i.e., pulling on the first end 500 to separate the fastener 330 from the body 302 would tear the body 302. The first adhesive may include an epoxy adhesive, cyanoacrylate, a polyurethane adhesive, an acrylic adhesive, a hot melt adhesive, a silicone adhesive, a rubber-based adhesive, or methyl methacrylate, or any combination thereof.

The second end 502 of the fastener 330 may include a second adhesive that enables the second end 502 to be adhered to the body 302, but detachable from the body 302 to enable the first end 502 to be repositioned from a first location to a second location different than the first location on the body 302. The second adhesive may include a low-tack adhesive, a tacky glue, a foam tape, a spray adhesive, or double-sided tape, or any combination thereof.

The fastener 330 may further include a visual indicator 504 provided at the second end 502. The visual indicator 504 may be, for example, a sticker, and may provide the user with a visual indication as to where the user can detach the fastener 330 from the body 302 to enable the user to reposition the fastener 330 (i.e. the second end 502) on the body 302.

With reference now to FIGS. 3 and 6, the stowage cover 300 may further include an identification component 340 (shown in phantom in FIG. 3) operable to help assist in properly positioning the stowage cover 300 on the robotic arm 106 (FIG. 1), as will be explained in more detail below. In some embodiments, the identification component 340 may be positioned on the second lateral side 1000 (FIG. 10) of the body 302, such as being angularly offset by about 180° around the body 302 from a center point of the mesh vent assembly 314. In other embodiments, however, the identification component 340 may be angularly offset from the mesh vent assembly 314 by other angular magnitudes, without departing from the scope of the disclosure.

The identification component 340 may be designed to interface with a sensor of the robotic surgical system 100 (FIG. 1), such as a sensor 1100 (see FIG. 11) on the robotic arm 106 (FIG. 11), thereby signaling to a controller (not shown) of the robotic surgical system 100 that the stowage cover 300 is positioned over the robotic arm 106. The identification component 340 may comprise a metal plate, such as a metal (e.g. stainless steel) disc, and the sensor 1100 (FIG. 11) may include a Hall-effect sensor operable to sense the proximity of the metal disc. The controller of the robotic surgical system 100 may prevent a user from transitioning the robotic arm 106 to a stowed position beneath the table 104 (FIG. 1) unless the sensor 1100 senses the presence of the identification component 340.

The identification component 340 may be coupled to the body 302 with an adhesive, such as with two-sided tape (not shown) interposing the identification component 340 and the body 302 or with an adhesive film layer 342 disposed over the identification component 340 to encapsulate the identification component 340 between the body 302 and the adhesive film layer 342, or a combination thereof.

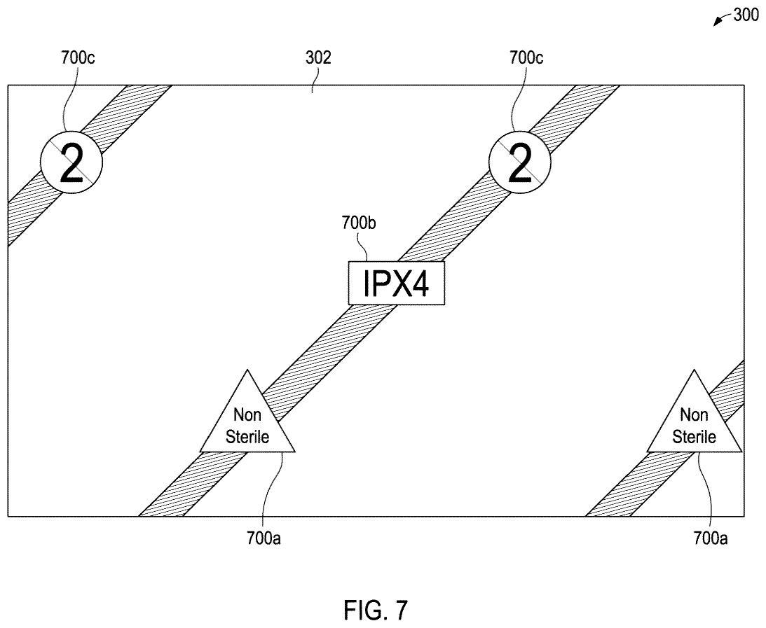

Referring now to FIG. 7, the body 302 of the stowage cover 300 may have printed thereon a plurality of symbols 700a, 700b, 700c. More specifically, the stowage cover 300 may be a non-sterile cover that satisfies IPX4 standards and that is intended for single use, accordingly, the printed symbols may convey to a user that the stowage cover 300 is non-sterile (700a), satisfies IPX4 standards (700b), and is intended for only a single use (700c). Alternatively, the stowage cover 300 may be a sterile cover and/or may be intended for more than one use.

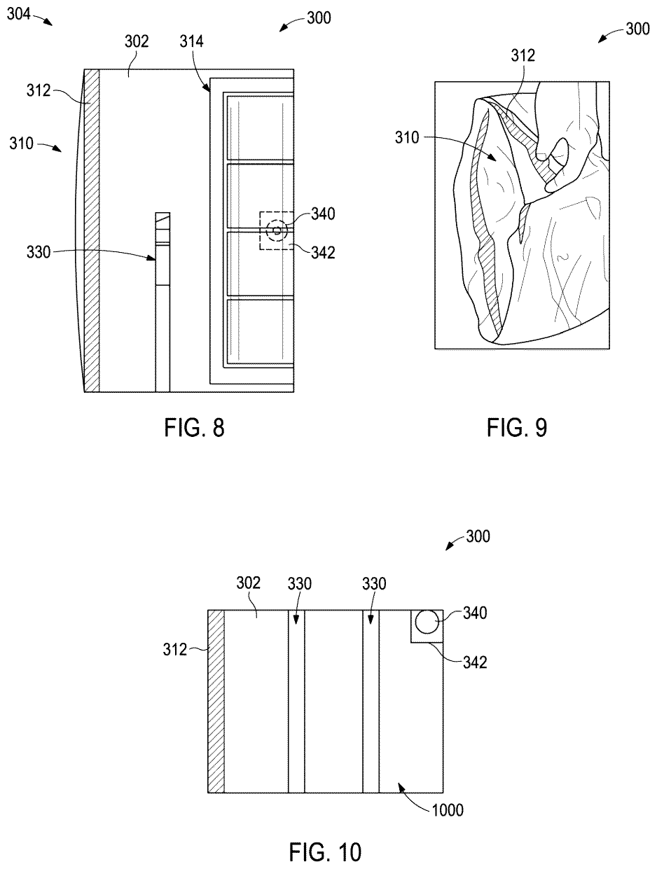

The stowage cover 300 may be transitionable between a plurality of folded and deployed states, or configurations. For instance, the stowage cover 300 may be transitionable between a deployed or fully unfolded state, as shown in FIG. 3, a partially-folded state, as shown in FIG. 8, and a fully folded state, as shown in FIG. 10. With reference to FIG. 3, the body 302 may be transitioned from the unfolded state to the folded state by incrementally and telescopically folding the body 302 at a plurality of axially-spaced fold locations, or edges 350. For example, a user may first open the opening 310, grasp the body 302 at the first (leftmost) folded edge 350 within the interior of the body 302 through the opening 310, and pull the first (leftmost) folded edge 350 to the proximal end 304, thereby defining a first folded portion within the interior of the body 302. The user may then, through the first folded portion, grasp the body at the second (second from the left) edge 350 and pull the second (second from the left) edge 350 through the first folded portion to the proximal end 304, thereby defining a second folded portion within the first folded portion. This folding process may then be repeated at subsequent folds locations 350 and at the visual indicator 302 until the stowage cover 300 transitions to a telescopically folded article, as shown in FIG. 8. In the partially-folded state, a user can at least partially open the opening 310 of the body 302 to gain access to the interior of the body 302, as shown in FIG. 9. To transition the stowage cover 300 from the partially-folded state to the folded state, the body 302 may be folded in half by rotating a first (bottom) edge into contact with a second, opposite (top) edge. In the folded state, the first lateral side 308 of the body 302 may be hidden (enveloped), thereby preventing damage to the mesh vent assembly 314 (FIG. 3) during transportation and distribution of the stowage cover 300.

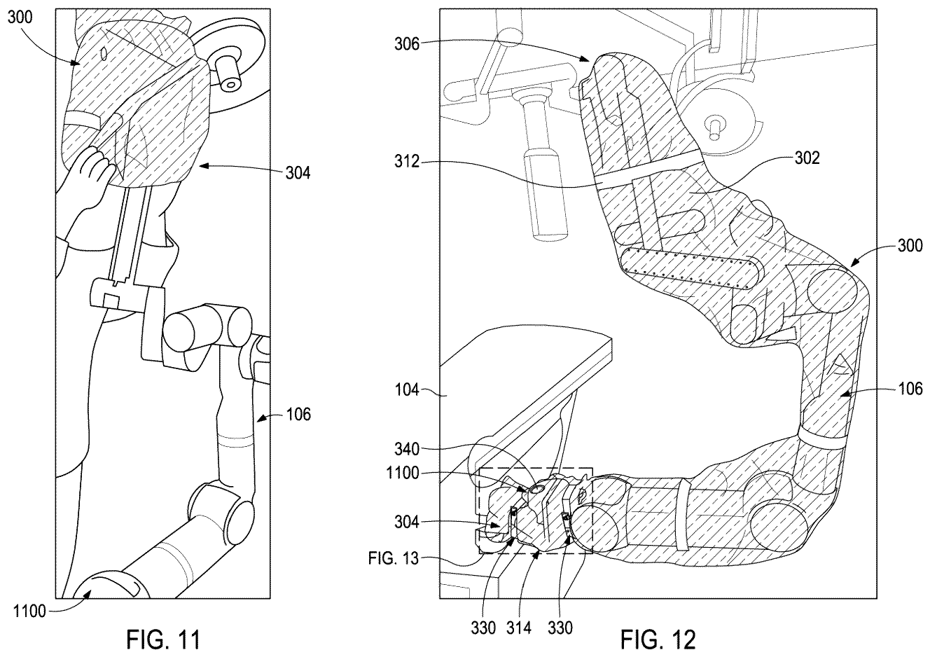

A method of positioning the stowage cover 300 over a robotic arm 106 (FIG. 1) will now be described. With reference to FIGS. 11 and 12, the robotic arm 106 may first be transitioned to a draping pose. As used herein, the term “draping pose” refers to a pose of the robotic arm 106 that is devoid (or substantially devoid) of acute angles between joints of the robotic arm 106, thereby providing a smooth path for a user to slide the stowage cover 300 over the robotic arm 106 and telescopically deploy the stowage cover 300.

Once the robotic arm 106 is transitioned to (placed in) the draping pose, a user then places the stowage cover 300 in the folded state at the distal (free) end of the robotic arm 106 such that the opening 310 (FIGS. 3 and 9) receives the distal (free) end of the robotic arm 106. The user can then advance the opening 310 along the robotic arm 106, thereby telescopically unfolding the stowage cover 300 such that the stowage cover incrementally transitions to the unfolded state. In the unfolded state, the proximal end 304 of the stowage cover 300 reaches (abuts) the table 104, as shown in FIG. 12.

With the stowage cover 300 in the unfolded state over the robotic arm 106, the user may rotate the stowage cover 300 relative to the robotic arm 106 to properly orient the stowage cover 300. More specifically, the stowage cover 300 may be rotated to position the identification component 340 at (against) the sensor 1100, which may be on an upper or “top” portion of the robotic arm 106, as seen in FIG. 11. Due to the angular offset between the identification component 340 and the mesh vent assembly 314 (FIG. 3), as described elsewhere herein, orienting the identification component 340 against the sensor 1100 may place the mesh vent assembly 314 into a confronting relationship with the floor. As such, fluid that may enter the stowage cover 300 via the opening 310, or any other locations on the stowage cover 300, would be drawn toward the mesh vent assembly 314 due to gravity, thereby allowing the fluid to exit the stowage cover 300 via the mesh vent assembly 314. Orienting the mesh vent assembly 314 towards the floor may also prevent, or at least substantially prevent, dust, dirt, and other debris from entering the stowage cover 300 via the mesh vent assembly 314.

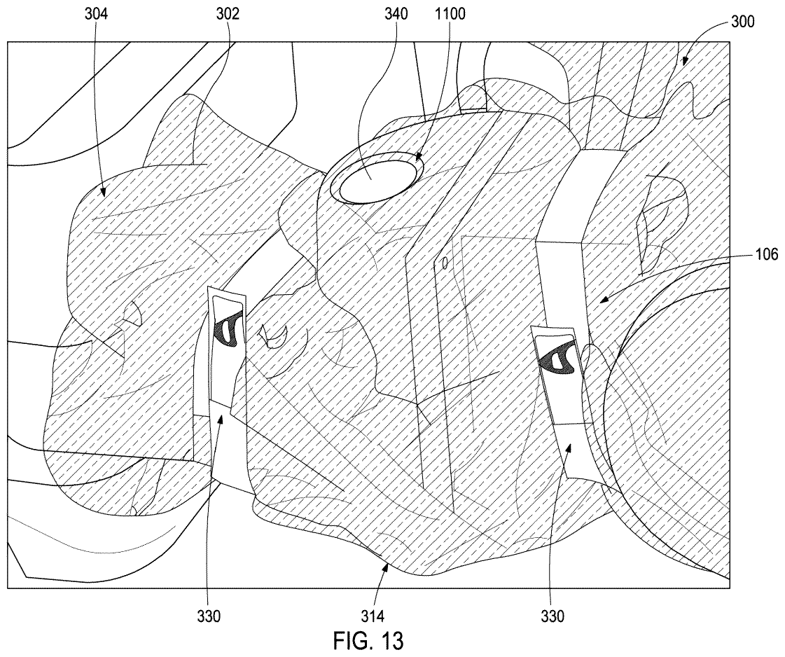

Once the identification component 340 is properly received at the sensor 1100, a signal is sent to a controller (not shown) of the robotic surgical system 100 (FIG. 1) indicating that the stowage cover 300 is properly positioned over (on) the robotic arm 106. At this point, the user may release the second ends 502 (FIG. 5) of the fasteners 330 from the body 302 and wrap the second ends 502 around the robotic arm 106, thereby tightening the body 302 against the robotic arm 106, as best seen in FIG. 13. The tightened fasteners 330 may help maintain the position of the stowage cover 300 relative to the robotic arm 106, while also preventing (or at least substantially preventing) fluid flow past the tightened fasteners 330 within the interior of the body 302.

Once the stowage cover 300 is secured to the robotic arm 106 with the fasteners 330, the robotic arm 106 may then be transitioned to a stowed state under the table 104. When it is desired to use the robotic arm 106 in a subsequent procedure, the process of removing the stowage cover 300 may be substantially similar to above, only completed in reverse.

Accordingly, the foregoing stowage cover 300 provides a protective barrier that is durable, functional, addresses specific needs of the robotic system 100 (FIG. 1), as well as improves safety, hygiene, and overall functionality of the robotic arms 106, such as when not in use.

Embodiments disclosed herein include:
A. A stowage cover comprising an elongate body including opposing proximal and distal ends, a first opening defined at the proximal end and sized to receive a robotic arm of a robotic surgical system into an interior of the body, a second opening defined in the body between the proximal and distal ends, and a fastener coupled to the body and operable to secure the body to the robotic arm once the body is received within the interior of the body.
B. A stowage cover comprising a body comprising an open proximal end, a closed distal end, a first lateral side, and a second lateral side opposite the first lateral side, a first opening defined at the open proximal end and sized to receive a robotic arm of a robotic surgical system, a second opening defined in the body on the first lateral side and between the proximal and distal ends, a mesh vent disposed over the second opening, and an identification component on the second lateral side, wherein the identification component is to interface with a sensor of the robotic surgical system, thereby signaling the presence of the stowage cover over the robotic arm.
C. A method of manufacturing a stowage cover for a robotic arm of a robotic surgical system, the method comprising providing a first opening in a body of the stowage cover at a proximal end thereof, the first opening being sized to receive the robotic arm, defining a second opening in the body between the proximal end and a distal end of the stowage cover, and coupling a fastener to the body, the fastener being operable to secure the stowage cover to the robotic arm when the robotic arm is received within an interior of the stowage cover.

Each of embodiments A-C may have one or more of the following additional elements in any combination: Element 1: wherein the fastener is a first fastener coupled to the body between the proximal end and the second opening, and wherein the stowage cover further comprises a second fastener coupled to the body between the distal end and the second opening and operable to secure the body to the robotic arm. Element 2: wherein the fastener comprises a first end secured to the body with a first adhesive and a second end secured to the body with a second adhesive different than the first adhesive, the second adhesive allowing the second end to be released from the body. Element 3: wherein the second opening comprises one or more slits extending along a portion of the body. Element 4: wherein the body is made of a polymer. Element 5: further comprising a mesh vent positioned over the second opening. Element 6: further comprising an identification component attached to the body and operable to interface with a sensor of the robotic surgical system, thereby signaling the presence of the stowage cover over the robotic arm. Element 7: wherein the identification component comprises a metal plate. Element 8: wherein the identification component comprises a metal plate. Element 9: further comprising a fastener coupled to the body and operable to secure the body to the robotic arm. Element 10: wherein the fastener is a first fastener coupled to the body between the proximal end and the second opening, and wherein the stowage cover further comprises a second fastener coupled to the body between the distal end and the second opening. Element 11: wherein the fastener comprises a first end secured to the body with a first adhesive and a second end secured to the body with a second adhesive different than the first adhesive, the second adhesive allowing the second end to be released from the body. Element 12: wherein the second opening comprises one or more slits extending along a portion of the body. Element 13: wherein the body is made of a polymer. Element 14: wherein coupling the fastener to the body comprises coupling a first fastener to the body between the proximal end and the second opening, the method further comprising coupling a second fastener to the body between the distal end and the second opening. Element 15: wherein coupling the fastener to the body comprises securing a first end of the fastener to the body with a first adhesive and securing a second end of the fastener to the body with a second adhesive different than the first adhesive, thereby allowing the second end to be released from the body. Element 16: further comprising coupling a mesh vent to the body over the second opening. Element 17: further comprising attaching an identification component to the body, the identification component being configured to interface with a sensor of the robotic surgical system, thereby signaling the presence of the stowage cover over the robotic arm when the robotic arm is received within the interior of the stowage cover.

By way of non-limiting example, exemplary combinations applicable to A, B, and C include: Element 1 with one or more of Elements 2-7; Element 2 with one or more of Elements 1 or 3-7; Element 3 with one or more of Elements 1, 2, and 4-7; Element 4 with one or more of Elements 1-3 and 5-7; Element 5 with one or more of Elements 1-4, 6, or 7; Element 6 with one or more of Elements 1-5 or 7; Element 8 with one or more of Elements 9-13; Element 9 with one or more of Elements 8 or 10-13; Element 12 with one or more of Elements 8-11; Element 13 with one or more of Elements 8-12; Element 14 with one or more of Elements 15-17;Element 15 with one or more of Elements 14, 16, or 17; Element 16 with one or more of Elements 14, 15, or 17; Element 17 with one or more of Elements 14-16.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

1. A stowage cover, comprising:
an elongate body including opposing proximal and distal ends;
a first opening defined at the proximal end and sized to receive a robotic arm of a robotic surgical system into an interior of the body;
a second opening defined in the body between the proximal and distal ends; and
a fastener coupled to the body and operable to secure the body to the robotic arm once the body is received within the interior of the body.

2. The stowage cover of claim 1, wherein the fastener is a first fastener coupled to the body between the proximal end and the second opening, and wherein the stowage cover further comprises a second fastener coupled to the body between the distal end and the second opening and operable to secure the body to the robotic arm.

3. The stowage cover of claim 1, wherein the fastener comprises:
a first end secured to the body with a first adhesive; and
a second end secured to the body with a second adhesive different than the first adhesive, the second adhesive allowing the second end to be released from the body.

4. The stowage cover of claim 1, wherein the second opening comprises one or more slits extending along a portion of the body.

5. The stowage cover of claim 1, wherein the body is made of a polymer.

6. The stowage cover of claim 1, further comprising a mesh vent positioned over the second opening.

7. The stowage cover of claim 1, further comprising an identification component attached to the body and operable to interface with a sensor of the robotic surgical system, thereby signaling the presence of the stowage cover over the robotic arm.

8. The stowage cover of claim 7, wherein the identification component comprises a metal plate.

9. A stowage cover, comprising:
a body comprising an open proximal end, a closed distal end, a first lateral side, and a second lateral side opposite the first lateral side;
a first opening defined at the open proximal end and sized to receive a robotic arm of a robotic surgical system;
a second opening defined in the body on the first lateral side and between the proximal and distal ends;
a mesh vent disposed over the second opening; and
an identification component on the second lateral side, wherein the identification component is to interface with a sensor of the robotic surgical system, thereby signaling the presence of the stowage cover over the robotic arm.

10. The stowage cover of claim 9, wherein the identification component comprises a metal plate.

11. The stowage cover of claim 9, further comprising a fastener coupled to the body and operable to secure the body to the robotic arm.

12. The stowage cover of claim 11, wherein the fastener is a first fastener coupled to the body between the proximal end and the second opening, and wherein the stowage cover further comprises a second fastener coupled to the body between the distal end and the second opening.

13. The stowage cover of claim 11, wherein the fastener comprises:
a first end secured to the body with a first adhesive; and
a second end secured to the body with a second adhesive different than the first adhesive, the second adhesive allowing the second end to be released from the body.

14. The stowage cover of claim 9, wherein the second opening comprises one or more slits extending along a portion of the body.

15. The stowage cover of claim 9, wherein the body is made of a polymer.

16. A method of manufacturing a stowage cover for a robotic arm of a robotic surgical system, the method comprising:
providing a first opening in a body of the stowage cover at a proximal end thereof, the first opening being sized to receive the robotic arm;
defining a second opening in the body between the proximal end and a distal end of the stowage cover; and
coupling a fastener to the body, the fastener being operable to secure the stowage cover to the robotic arm when the robotic arm is received within an interior of the stowage cover.

17. The method of claim 16, wherein coupling the fastener to the body comprises coupling a first fastener to the body between the proximal end and the second opening, the method further comprising coupling a second fastener to the body between the distal end and the second opening.

18. The method of claim 16, wherein coupling the fastener to the body comprises:
securing a first end of the fastener to the body with a first adhesive; and
securing a second end of the fastener to the body with a second adhesive different than the first adhesive, thereby allowing the second end to be released from the body.

19. The method of claim 16, further comprising coupling a mesh vent to the body over the second opening.

20. The method of claim 16, further comprising attaching an identification component to the body, the identification component being configured to interface with a sensor of the robotic surgical system, thereby signaling the presence of the stowage cover over the robotic arm when the robotic arm is received within the interior of the stowage cover.
